(19) United States
(12) Patent Application Publication
(10) Pub. No.: US 2015/0099272 A1
(43) Pub. Date: Apr. 9, 2015

(54) APPARATUS FOR MEASURING FLOATING MICROORGANISMS IN A GAS PHASE IN REAL TIME USING A SYSTEM FOR DISSOLVING MICROORGANISMS AND ATP ILLUMINATION, AND METHOD FOR DETECTING SAME

(71) Applicants: INDUSTRY-ACADEMIC COOPERATION FOUNDATION, YONSEI UNIVERSITY, Seoul (KR); LG ELECTRONICS INC., Seoul (KR)

(72) Inventors: Jung Ho HWANG, Seoul (KR); Chul Woo PARK, Seoul (KR); Ji-Woon PARK, Seoul (KR); Jae Won CHANG, Seoul (KR); Sung Hwa LEE, Seoul (KR); Bong-Jo SUNG, Seoul (KR)

(21) Appl. No.: 14/562,690

(22) Filed: Dec. 6, 2014

Related U.S. Application Data
(62) Division of application No. 13/982,056, filed on Oct. 7, 2013.

(51) Int. Cl. C12Q 1/04

(52) U.S. Cl. CPC C12Q 1/04
USPC 435/34

(57) ABSTRACT

The present invention relates to a method for measuring airborne microorganisms in real time using a microorganism lysis system and ATP bioluminescence, the method including sampling the airborne microorganisms in a particle classification device to which an ATP-reactive luminescent agent is applied and, at the same time, lysing the microorganisms in a microorganism lysis system under continuous operation to extract adenosine triphosphate (ATP) of the microorganisms sampled in the particle classification device, thus inducing a luminescent reaction between the ATP-reactive luminescent agent and the ATP of the particle classification device in real time; and measuring the concentration of microorganisms using a light receiving device. According to the detection method using ATP organism illumination, the floating microorganisms in the gas phase can be readily detected and the detection can be automatically conducted in real time without manual labor.

### CROSS REFERENCE TO RELATED APPLICATION

This application is a division of U.S. patent application Ser. No. 13/982,056, filed Oct. 7, 2013, which was the National Stage of International Application No. PCT/KR2011/007217, filed Sep. 30, 2011, the entire contents of which are incorporated herein by reference.

### BACKGROUND OF THE INVENTION

1. Technical Field

The present invention relates to an apparatus and method for measuring airborne microorganisms and, more particularly, to an apparatus and method for measuring airborne microorganisms in real time, which can rapidly measure microorganisms present in the air using an ATP bioluminescence method.

2. Description of the Related Art With the recent emergence of avian influenza, new influenza, etc., the problem of airborne infection arises, and thus the measurement of airborne microorganisms is considered important, together with the rapid growth of a biosensor market.

Conventional methods for measuring airborne microorganisms include a culture method of sampling biogenic particles suspended in a gas sample on the surface of a solid or liquid suitable for their growth to be cultured in, an appropriate temperature and humidity environment and calculating the number of collected microorganisms from the number of colonies present on the surface, a staining method using a fluorescence microscope after staining, etc.

With the recently developed ATP bioluminescence method which uses the principle that adenosine triphosphate (ATP) and luciferin-luciferase react to emit light, a series of processes of ATP destruction, ATP extraction, and luminescence measurement can be performed within about 30 minutes.

However, according the above methods, it is impossible to measure the microorganisms present in the air in real time, and a series of manual operations including a separate sampling process, a pretreatment process, etc. are required, which makes it difficult to develop a system for automatically measuring the airborne microorganisms using these methods.

In practice, the existing biosensors require a separate sampling process to measure the airborne microorganisms, which takes a minimum of 20 minutes and a maximum of 2 hours. Moreover, there is a UV-APS of TSI Inc. for the measurement without a separate sampling process, which is very expensive, around 200 million Korean won, and is thus used by some professional research institutions and cannot be widely used

Further, an ATP extracting agent is basically required to apply the ATP bioluminescence method, but if the ATP extracting agent is used in the system for measuring the airborne microorganisms, it may have adverse effects on the body such as toxicity. In addition, it is necessary to continuously supply the ATP extracting agent for the application of an automatic system, but the continuous supply of commercially available ATP extracting agents increases the cost burden.

### SUMMARY OF THE INVENTION

Accordingly, the present invention has been made to solve the above-described problems, and an object of the present invention is to provide an apparatus and method for measuring airborne microorganisms, which can rapidly measure the airborne microorganisms using an ATP bioluminescence method without a series of manual operations, thus enabling real-time automatic measurement and achieving safety and low costs.

To accomplish the above objects of the present invention, an, aspect of the present invention provides an apparatus for measuring airborne microorganisms in real time using a microorganism lysis system and ATP bioluminescence, the apparatus comprising: a particle classification device 10 in which airborne microorganisms are collected and to which an ATP-reactive luminescent agent is applied; a microorganism lysis system 20 which extracts adenosine triphosphate (ATP) by lysing the microorganisms; and a light receiving device 30 which detects light generated by reaction between the ATP extracted by the is microorganism lysis system 20 and the ATP-reactive luminescent agent applied to the particle classification device 10.

Here, the particle classification device may comprise any one selected from the group consisting of an electrostatic precipitator, an inertial impactor, a cyclone, and a centrifuge.

Moreover, the airborne microorganisms may preferably be collected on a collecting plate or in a collecting space provided in the particle classification device 10 and may be collected in a liquid applied to the collecting plate of the particle classification device 10 or accommodated in the collecting space.

Furthermore, the particle classification device 10 in a state where the ATP-reactive luminescent agent is absorbed may preferably be installed or the apparatus of the present invention may further comprise an ATP-reactive luminescent agent supply device 11 which supplies the ATP-reactive luminescent agent to the particle classification device 10.

In addition, the ATP-reactive luminescent agent may preferably be luciferin. Additionally, the particle classification device 10 may preferably have a collection efficiency of more than 50% for particles of 1 μm in size.

Moreover, the microorganism lysis system 20 may preferably be an ion generator which extracts the APT by damaging cell walls of microorganisms due to a repulsive force between charged ions attached to the microorganisms.

Here, the ion generator may preferably be an ozone-free ion generator which uses a carbon brush in which the diameter of a discharge tip is less than 10 μm.

Furthermore, the microorganism lysis system 20 may preferably be a plasma discharger which extracts the ATP by damaging cell walls of microorganisms due to collision of ions or electrons in high concentration generated by high voltage discharge.

In addition, the light receiving device 30 may preferably have a sensitivity capable of detecting light in a wavelength band of 400 nm to 700 nm.

Additionally, the apparatus of the present invention may further comprise a microbial concentration calculation unit 61 which converts an electrical signal output from the light receiving device 30 into numerical data to output the concentration of microorganisms or the level of contamination as a specific number depending on the correlation with a bioluminescence value proportional to the concentration of microorganisms.

Moreover, the apparatus of the present invention may further comprise a display device 40 which displays in real time the concentration of microorganisms or the level of contamination measured by the light detected by the light receiving device 30.

Furthermore, the apparatus of the present invention may further comprise a wireless controller 64 which comprises a calculation unit 62 which determines whether the concentration of microorganisms or the level of contamination exceeds a predeterrnined value and an output unit 65 which wirelessly transmits a control signal to an external air conditioning device 70 such as an air purifier or ventilator or to an external device which comprises a wireless communication device 80 such as a portable terminal when it is determined that the concentration of microorganisms or the level of contamination exceeds the predetermined value.

In addition, the apparatus of the present invention may further comprise a communication unit 63 which wirelessly transmits information about the concentration of microorganisms or the level of contamination measured by the light detected by the light receiving device 30 to the wireless communication device 80, and the wireless communication device 80 may comprise a receiving unit 81 which wirelessly receives a signal from the communication unit 63 and a signal processing unit 82 which converts the signal of the receiving unit 81 into information about the concentration of microorganisms or the level of contamination and displays the information on the corresponding wireless communication device 80.

Additionally, the apparatus of the present invention may further comprise a flow generating means 50 which is configured to forcibly flow air toward the particle classification device 10, thus creating a pressure difference. Meanwhile, another aspect of the present invention provides a method for measuring airborne microorganisms in real time using a microorganism lysis system and ATP bioluminescence, the method comprising the steps of: sampling the airborne microorganisms in a particle classification device 10 to which an AlP-reactive luminescent agent is applied and, at the same time, lysing the microorganisms in a microorganism lysis System 20 under continuous operation to extract adenosine triphosphate (ATP) of the microorganisms sampled in the particle classification device 10, thus inducing a luminescent reaction between the ATP-reactive luminescent agent and the ATP of the particle classification device 10 in real time; and measuring the concentration of microorganisms using a light receiving device 30.

Moreover, still another aspect of the present invention provides a method for measuring airborne microorganisms in real time using a microorganism lysis system and ATP bioluminescence, the method comprising: a microorganism collection step of collecting the microorganisms in a particle classification device 10; an ATP extraction step of extracting adenosine triphosphate (ATP) by lysing the microorganisms by operating a microorganism lysis system 20; and a real-time detection step of measuring in real time, at a light receiving device 30, light generated by reaction between the ATP extracted in the ATP extraction step and an ATP-reactive luminescent agent present in the particle classification device 10.

Here, the method of the present invention may further comprise a real-time display step of converting data detected by the light receiving device 30 in the real-time detection step into the concentration of microorganisms and displaying the concentration of microorganisms in real time.

### BRIEF DESCRIPTION OF THE DRAWINGS

FIG. 1 is a conceptual diagram showing an apparatus for measuring airborne microorganisms in real time using a microorganism lysis system and ATP bioluminescence in accordance with a first embodiment of the present invention.

FIG. 2 is a conceptual diagram showing an apparatus for measuring airborne microorganisms in real time using a microorganism lysis system and ATP bioluminescence in accordance with a second embodiment of the present invention.

FIGS. 3A to 3C are conceptual diagrams showing various embodiments of a particle classification device.

FIG. 4 is a graph showing the measurement results of airborne microorganisms according to the operation time.

FIG. 5 is a flowchart showing a method for measuring airborne microorganisms in real time using a microorganism lysis system and ATP bioluminescence according to the present invention.

### DESCRIPTION OF THE PREFERRED EMBODIMENTS

Hereinafter, an apparatus and method for measuring airborne microorganisms in real time using a microorganism lysis system and ATP bioluminescence according to the present invention will be described with reference to the accompanying drawings.

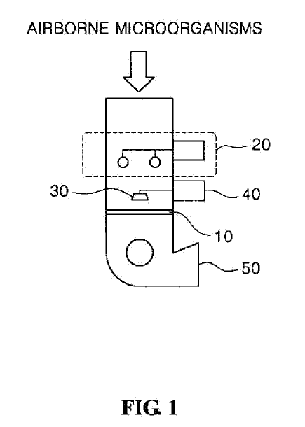

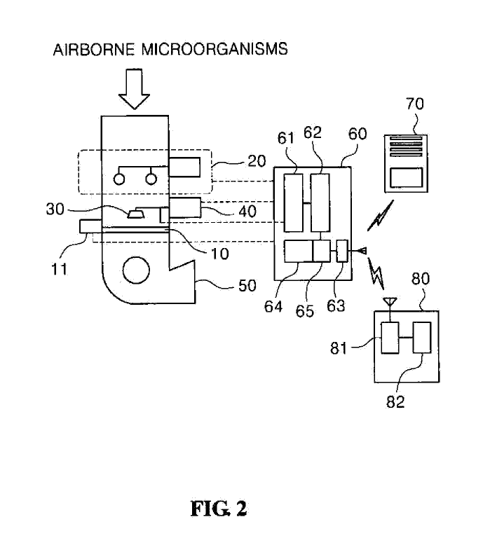

An apparatus for measuring airborne microorganisms in real time using a microorganism lysis system and ATP bioluminescence according to the present invention generally comprises a particle classification device 10, a microorganism lysis system 20, and a light receiving device 30 as shown in FIGS. 1 and 2, in which the airborne microorganisms are sampled in the particle classification device 10 and, at the same time, the microorganisms are continuously lysed by the microorganism lysis system 20 (which will be described in detail later) to extract adenosine triphosphate (ATP), thus automatically measuring bioluminescence.

While the particle classification device 10 is shown in the form of a flat plate in FIGS. 1 and 2, only a component corresponding to a collecting plate (which will be described in detail later) is conceptually shown to explicitly represent the interaction between the particle classification device 10, the microorganism lysis system 20, and the light receiving device 30, and the shape and structure of the particle classification device 10 is not particularly limited. Moreover, the particle classification device 10 can be applied in various embodiments, which will be described below.

The particle classification device 10 generally refers to, a dust collector or filter system, such as an electrostatic precipitator, an inertial impactor, a cyclone, a centrifuge, etc., which comprises a collecting plate or collecting space capable of collecting airborne particles by a solid sampling method or liquid sampling method.

The electrostatic precipitator is a dust collector that uses the electrostatic principle that a corona discharge occurs when a negative (−) voltage (or positive (+) voltage) is applied to a discharge electrode from a high DC voltage source, and negative (−) ions (or positive (+) ions) generated at this time are charged to airborne dust particles, which are then moved by an electric force to a dust collecting electrode (collecting plate) receiving a positive (+) voltage (or negative (−) voltage) and collected therein.

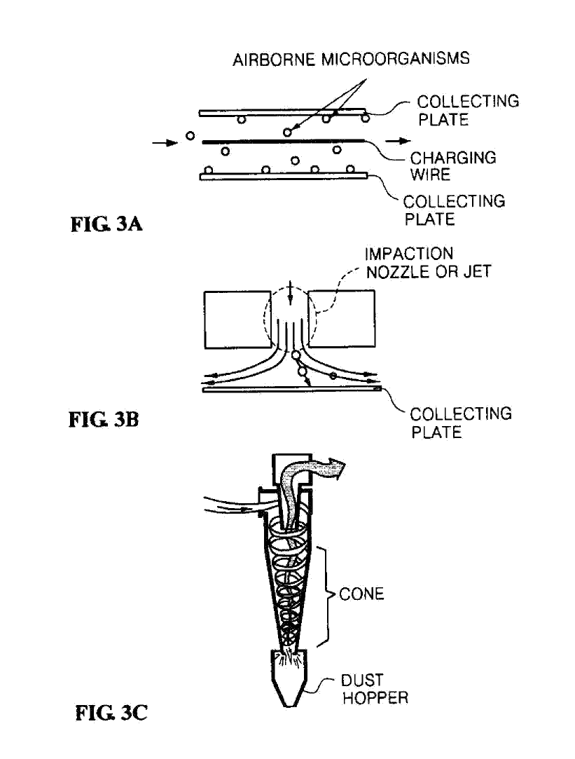

FIG. 3A shows an example of a wire-to-plate type electrostatic precipitator, which are most widely used among various types of electrostatic precipitators, in which an electric field is generated between a charging wire and a collecting plate, and particles charged by passing through the charging wire and the collecting plate are collected on the, collecting plate.

The inertial compactor has a structure in which an impaction plate or receiving tube (hereinafter collectively referred to as the “collecting plate”) is provided below an acceleration nozzle (or impaction nozzle).

FIG. 3B shows an example of the inertial impactor in which the flow direction of air passing through the acceleration nozzle or jet is changed 90° by the collecting plate. At this time, the flow direction of particles above a predetermined mass among the particles contained in the air is not completely changed by inertia, and the particles impact on the collecting plate and are then collected therein.

The cyclone is one of the separators using centrifugal force, which are widely used to separate solid particles from a fluid or to separate liquid droplets from a gas stream, and has various types and specifications, and FIG. 3C shows an example of the cyclone.

The air containing particles is tangentially introduced into a circular cyclone and swirls along a cylindrical inner wall to create a swirling flow. This swirling flow is continuously maintained up to a cone area at the bottom of the cyclone to push the particles toward the inner wall by centrifugal force to be separated from the flow. The flow (air) from which the particles are removed rises upward from the bottom of the cone and is then discharged through an outlet, and the separated particles drop along the inner wall of the cone and are then collected in a dust hopper (hereinafter collectively referred to as the “collecting plate”).

The centrifuge is a device that utilizes continuous centrifugal force generated by high speed rotation. Although the cyclone is also a separator using centrifugal force, the centrifuge can separate particles contained in the air toward the outer wall of a rotating vessel using the rotating vessel rotating at high speed, compared to the cyclone.

The electrostatic precipitator is suitably applied to a large volume or high flow due to its low pressure loss and has high dust collection efficiency for nano-sized fine particles (less than 100 nm). Compared to this, the inertial impactor, the cyclone, etc. have advantages of low production cost and maintenance cost due to their simple structures. The solid sampling method is to sample a material to be measured in a solid by adsorption, reaction, etc. in which an air sample is passed through a particle layer of the solid to be absorbed. This solid sampling method can be applied, in a process of sampling airborne microorganisms on the collecting plate or in a collecting space provided in the particle classification device 10.

The liquid sampling method is to sample a material to be measured in a liquid by dissolution, reaction, precipitation, suspension, etc. in which an air sample is passed through the liquid or brought into contact with the surface of the liquid. The type of absorbent liquid varies depending on the type of material to be sampled.

A liquid may be applied on the collecting plate or accommodated in the collecting space, and the airborne microorganisms may be sampled by the liquid sampling method.

Besides, a filtration sampling method of sampling a material to be measured in a filter medium by passing an air sample through the filter medium using the particle classification device 10, a cooling condensation sampling method of sampling a material to be measured by bringing an air sample into contact with a cooled pipe to be condensed, a direct sampling method of sampling a material to be measured by directly sampling an air sample in a collecting bag, a collecting bottle, a vacuum collecting bottle, or a syringe without dissolving, reacting, or adsorbing the air sample, a diffusion sampling method of sampling and analyzing an air sample using the principle of molecular diffusion, etc. may be employed.

Microorganisms present in the air are collected in the particle classification device 10 while passing therethrough, and an ATP-reactive luminescent agent required for bioluminescence is absorbed into the particle classification device 10 or the ATP-reactive luminescent agent is continuously or intermittently supplied to the particle classification device 10.

In order to maintain the ATP-reactive luminescent agent present in the particle classification device 10, the particle classification device 10 in a state where the ATP-reactive luminescent agent is already applied or absorbed may be installed as shown in FIG. 1, or an ATP-reactive luminescent agent supply device 11 for injecting or supplying a required amount of ATP-reactive luminescent agent to the particle classification device 10 may be provided separately from the particle classification device 10 as shown in FIG. 2.

In general, visible pollen, mold, microbes, fiber dust, etc. have a particle size of more than 100 μm, and bacteria have a size of 0.1 μm to 100 μm. Therefore, it is preferred to select a particle classification device 10 having a collection efficiency of more than 50% for particles of 1 μm in size in view of the adequacy of the collection efficiency such as pressure loss, initial investment cost, maintenance cost, etc.

The ATP-reactive luminescent agent supply device 11 is not limited to a specific structure and form as long as it can supply a liquid ATP-reactive luminescent agent to the particle classification device 10. Moreover, it is preferred to apply a more appropriate device in terms of overall conditions such as use environment, specification, etc. of well-known liquid supply devices, and thus a detailed description thereof will be omitted.

The microorganism lysis system 20 generally refers to a component that extracts adenosine triphosphate (ATP), DNA, RNA, etc. present in the microorganisms collected in the particle classification device 10 using ions, electromagnetic force of electrons, antimicrobial materials, thermal energy, catalyst, etc. or obtained by lysing the microorganisms moving toward the particle classification device 10. Here, the lysis of microorganisms means that a single microorganism is degraded into several elements or extracted into several elements, instead of dissolving the microorganism into a liquid state.

When the microorganism lysis system 20 is configured as an ion generator, the larger the diameter of a discharge tip provided in the, ion generator, the larger the power consumption, and when the power consumption is high, ozone that is harmful to the human body can even be generated as well as the ions. Therefore, it is preferred to apply an ozone-free ion generator which uses a carbon brush in which the diameter of the discharge tip is less than 10 μm.

According to the ozone-free ion generator which uses the carbon brush in which the diameter of the discharge tip is less than 10 μm, it has a low power consumption of less than 4 W, and thus ozone in a concentration of less than 0.01 ppm is generated. Therefore, it can stably meet the ozone standard level of 0.06 ppm specified under the Guideline for the Management of Office Air Quality and Article 27(1) of the Industrial Safety and Health Act.

When the microorganism lysis system 20 is configured as an ion generator, the ATP is extracted by damaging cell walls of microorganisms due to a repulsive force between charged ions attached to the microorganisms, whereas when the microorganism lysis system 20 is configured as a plasma discharger, the ATP is extracted by damaging cell walls of microorganisms due to collision of ions or electrons in high concentration generated by high voltage discharge.

The ATP extracted by the microorganism lysis system 20 is exposed to the outside of the cells of the microorganisms and, at the same time, reacts with the ATP-reactive luminescent agent in the particle classification device 10 to generate light. Then, the light receiving device 30 which converts light into electricity, such as a photodiode (PD), an avalanche photodiode (APD), etc., detects the light generated by ATP bioluminescence, thus measuring the concentration of microorganisms or the level of contamination.

All organisms store energy generated by the oxidation of organics in a compound called ATP and hydrolyze the ATP to sustain activity and maintain body temperature, if necessary, using the energy generated during the hydrolysis. This ATP generates bioelectricity and causes bioluminescence.

The light receiving device 30 is an element that measures photon flux or optical power by converting the energy of the absorbed photons into a measurable form. The light receiving device 30 has advantages of high sensitivity at the operating wavelengths, high response speed, and minimum noise and is thus widely used as an photodetector for detecting an optical signal in an optical fiber communication system operating in the near-infrared region (0.8-1.6 μm).

In particular, a photoelectric detector, one of the light receiving devices, is based on the photoeffect in which a carrier such as an electron, hole, etc. is generated in a material forming the detector by the photons absorbed in the detector, and a measurable current is generated by the flow of the carrier.

The wavelengths of light as electromagnetic waves discernible by the human eye are in the range of 380 nm to 780 nm. As monochromatic lights, violet-blue light has wavelengths of 400-500 nm, blue light has wavelengths of 450-500 nm, green light has wavelengths of 500-570 nm, yellow light has wavelengths of 570-590 nm, orange light has wavelengths of 590-610 nm, and red light has wavelengths of 610-700 nm, and the light receiving device 30 has a sensitivity capable of detecting light in a wavelength band of 400 nm to 700 nm.

When the particle classification device 10 collects airborne microorganisms, a pressure difference is created by means of a flow generating means 50 such as a blower or pump to forcibly flow air on one side with respect to the particle classification device 10 to the other side. Here, the microorganism lysis system 20 and the light receiving device 30 are installed on a path through which the air flows to the particle classification device 10, i.e., on one side of the particle classification device 10, and the flow generation means 50 is installed on the other side of the particle classification device 10.

The higher the concentration of microorganisms, the larger the amount of ATP extracted, and the higher the level of light intensity. The light receiving device 30 converts the received light into an electrical signal such as a voltage, current, and frequency and outputs the electrical signal. Moreover, a microbial concentration calculation unit 61 provided in a controller converts the electrical signal input from the light receiving device 30 into numerical data such that the concentration of microorganisms or the level of contamination can be output as a specific number depending on the correlation with a bioluminescence value proportional to the concentration of microorganisms.

The light detected by the light receiving device 30 is converted into numerical data by the microbial concentration calculation unit 61, and a display device 40 displays the concentration of microorganisms or the level of contamination in real time based on the numerical data.

A wireless controller 64, which comprises a calculation unit 62 which determines whether the concentration of microorganisms or the level of contamination exceeds a predetermined value and an output unit 65 which is connected to a communication unit 63 which wirelessly transmits a control signal to an external air conditioning device 70 such as an air purifier or ventilator when it is determined by the calculation unit 62 that the concentration of microorganisms or the level of contamination exceeds the predetermined value, may be used.

With the use of the wireless controller 64, it is possible to manage the main body of the apparatus for measuring airborne microorganisms in accordance with an embodiment of the present invention (including the particle classification device 10, the microorganism lysis system 20, and the light receiving device 30) in conjunction with the air purifier or ventilator which are independently provided in different spaces.

For example, if the air is contaminated to the extent that the concentration of airborne microorganisms in the space where the main body of the apparatus for measuring airborne microorganisms exceeds the predetermined value, it is possible to automatically operate the air purifier or ventilator using the wireless controller 64 to maintain the level of air quality above a predetermined level.

Moreover, the communication unit 63 can wirelessly transmit information about the concentration of microorganisms or the level of contamination measured by the light detected by the light receiving device 30 to a wireless communication device 80 such as a portable terminal. The wireless communication device 80 may comprise a receiving unit 81 which wirelessly receives a signal from the communication unit 63 and a signal processing unit 82 which converts the signal of the receiving unit 81 into information about the concentration of microorganisms or the level of contamination and displays the information.

Therefore, a user or manager carrying the wireless communication device 80 can identify a variety of information related to the air quality using the wireless communication device 80 without having to move to the main body of the apparatus for measuring airborne microorganisms at any time when the user or manager wants to identify the level of contamination of airborne microorganisms. Furthermore, the user or manager can directly operate the air purifier or ventilator from a remote place by remotely connecting the wireless communication device 80 to the wireless controller 64 through the communication unit 63.

Bioluminescence is a kind of photochemical reaction in which the energy generated when a certain organic compound is oxidized by enzymatic reaction is emitted in the form of light energy to the outside of the body. In detail, luciferin, a luminescent material, is combined with ATP to form a luciferin-ATP complex, thus generating two inorganic phosphorus molecules (H3PO4). Here, the luciferin is a reduced type and is thus expressed as LH2 (LH2+ATP→LH2−AMP+2H3PO4).

LH₂+ATP generated in the above reaction are oxidized by reaction with oxygen and turned into an unstable energy state, and thus the oxidized product in an unstable state is immediately degraded to generate oxidized luciferin and AMP, thus generating light (hv). Here, L represents the oxidized luciferin, and L-AMP* represents the luciferin-AMP complex in an unstable energy state (LH₂−AMP+½ O2→L−AMP*+H2O)(L-AMP*→L+AMP+hv (light energy)).

The process in which LH2-AMP are oxidized by reaction with oxygen (½ O2) is achieved by the catalytic action of an enzyme, and thus the bioluminescence occurs in the presence of luciferin, ATP, luciferase, and oxygen. Here, it is calculated that one light quantum is emitted by the oxidation of one luciferin molecule.

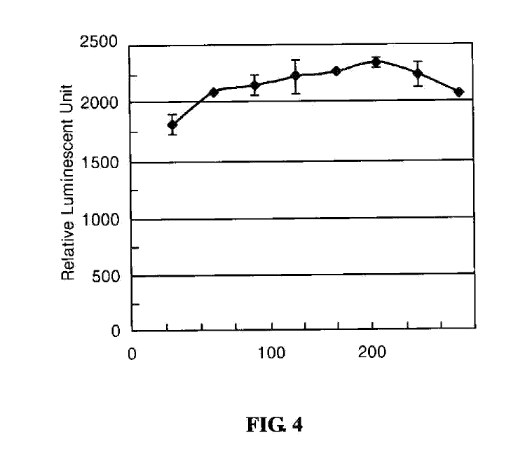

When the ATP-reactive luminescent agent is configured as luciferin, it is possible to rapidly measure the airborne microorganisms within five minutes by the above-described process. The graph shown in FIG. 4 shows the change in measured values of airborne microorganisms according to the operation time of the apparatus for measuring airborne microorganisms in accordance with a first embodiment of the present invention as shown in FIG. 1, from which it can be seen that the maximum light intensity is measured within three minutes (180 sec), implying that a measurement time of three minutes is required.

In the experiment in the graph shown in FIG. 4, an ozone-free ion generator was used as the microorganism lysis system 20, and the experiment was carried out at an air flow rate of 3 l/min, at a temperature of 23° C., at an ion density of 9×10⁶ number/cm³, and at a bioaerosol concentration of 93000 CFU/m³, and the light intensity is expressed in relative luminescence unit (RLU).

A method for measuring airborne microorganisms in real time using a microorganism lysis system and ATP bioluminescence according to the present invention relates to a method for automatically measuring the concentration of microorganisms in real time using the apparatus for measuring airborne microorganisms in real time having the above-described configuration according to the present invention.

Airborne microorganisms are sampled in the particle classification device 10 into which luciferin is absorbed and, at the same time, the microorganisms are lysed by the microorganism lysis system 20 under continuous operation. Then, adenosine triphosphate (ATP) of the microorganisms collected in the particle classification device 10 is extracted to induce a luminescent reaction between the luciferin and the ATP of the particle classification device 10 in real time, thus measuring the concentration of microorganisms using the light receiving device 30.

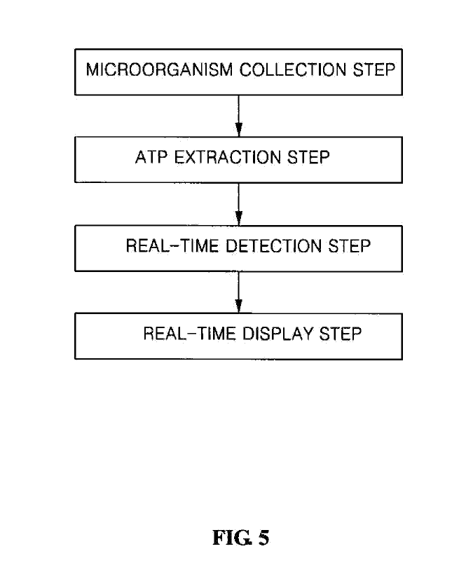

As shown in FIG. 5, a microorganism collection step, an ATP extraction step, a real-time detection step, and a real-time display step are sequentially performed. However, the overall processes are performed within a short time such as five minutes, and each step is continuously performed by each component, thus providing an effect that the overall processes are simultaneously performed.

In the microorganisms collection step, the airborne microorganisms are collected in the particle classification device 10, and in the ATP extraction step, the microorganism lysis system 20 is operated to lyse the microorganisms collected in the particle classification device 10, thus extracting adenosine triphosphate (ATP).

In the real-time detection step, the intensity of light generated by the reaction between the ATP extracted in the ATP extraction step and the luciferin present in the particle classification device 10 is measured in real time by the light receiving device 30, and in the real-time display step, the data detected by the light receiving device 30 in the real-time detection step is converted into the concentration of microorganisms, thus displaying the concentration of microorganisms in real time.

According to the apparatus and method for measuring airborne microorganisms using the microorganism lysis system and ATP bioluminescence having the above-described configuration according to the present invention, airborne microorganisms are sampled in the particle classification device 10 into which luciferin is absorbed and, at the same time, the microorganisms are lysed by the microorganism lysis system 20 under continuous operation to extract the ATP of the microorganisms collected in the particle classification device 10, thus inducing a luminescent reaction between the ATP-reactive luminescent agent and the ATP of the particle classification device 10 in real time.

The existing ion generators, plasma dischargers, and their related techniques are used only to remove toxic substances such as bioaerosols, particles, gases, and the existing methods to lyse microorganisms are limited to the user of reagents such as lysis-buffer. However, in the present invention, a semi-permanently usable device such as an ion generator, plasma discharger, etc. is applied to the microorganism lysis system.

Therefore, it is possible to rapidly measure the microorganism present in the air within five minutes by the ATP bioluminescence method. Moreover, the processes from the microorganism sampling, the ATP extraction, and the bioluminescence are automatically performed without a series of manual operations, thus enabling real-time automatic measurement of airborne microorganisms.

With the application of a semi-permanently usable device such as an ion generator, plasma discharger, etc. to the microorganism lysis system, the apparatus of the present invention can be safely used at low cost and simply controlled by an electrical method without the high costs required to continuously supply and control the reagents such as lysis-buffer the lysis of microorganisms, the difficulties in management and maintenance, and the toxicity to the human body.

The existing biosensors are expensive and require a series of manual operations, resulting in an increase in manpower and cost. However, according to the present invention, it is possible to enable real-time automatic measurement of airborne microorganisms with low cost and safety, and thus it is possible to allow the apparatus for measuring the airborne microorganisms in real time to be widely and commonly used.

Therefore, it is possible to simply detect mad cow disease, swine fever, avian influenza, etc. in stock farms and food plants or measure the growth of harmful microorganisms in food, and thus it is possible to effectively prevent social and economic losses due to airborne infection. Moreover, it is possible to meet the demand by the rapidly growing biosensor market, thus contributing to the improvement of human welfare due to an increased use of biosensors.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. A method for measuring airborne microorganisms in real time using a microorganism lysis system and ATP bioluminescence, the method comprising:
sampling the airborne microorganisms in a particle classification device (10) to which an ATP-reactive luminescent agent is applied and, at the same time, lysing the microorganisms in a microorganism lysis system (20) under continuous operation to extract adenosine triphosphate (ATP) of the microorganisms sampled in the particle classification device (10), thus inducing a luminescent reaction between the ATP-reactive luminescent agent and the ATP of the particle classification device (10) in real time; and
measuring the concentration of microorganisms using a light receiving device (30).

2. A method for measuring airborne microorganisms in real time using a microorganism lysis system and ATP bioluminescence, the method comprising:
a microorganism collection step of collecting the microorganisms in a particle classification device (10);
an ATP extraction step of extracting adenosine triphosphate (ATP) by lysing the microorganisms by operating a microorganism lysis system (20); and
a real-time detection step of measuring in real time, at a light receiving device (30), light generated by reaction between the ATP extracted in the ATP extraction step and an ATP-reactive luminescent agent present in the particle classification device (10).

3. The method of claim 2, further comprising a real-time display step of converting data detected by the light receiving device (30) in the real-time detection step into'the concentration of microorganisms and displaying the concentration of microorganisms in real time.
